(19) United States
(12) Patent Application Publication
(10) Pub. No.: US 2016/0347681 A1
(43) Pub. Date: Dec. 1, 2016

(54) ION-EXCHANGED ZSM-5 ZEOLITE CATALYST FOR CONVERSION OF ALKYL HALIDE TO OLEFINS

(71) Applicant: SABIC Global Technologies B.V., 4612 PX Bergen op Zoom (NL)

(72) Inventors: Ashim Kumar Ghosh, Houston, TX (US); Edouard Mamedov, Houston, TX (US); Neeta Kulkarni, Houston, TX (US); Armando Araujo, Sugar Land, TX (US); Mike Mier, Waller, TX (US)

(21) Appl. No.: 15/153,873

(22) Filed: May 13, 2016

Related U.S. Application Data
(60) Provisional application No. 62/167,030, filed on May 27, 2015.

(51) Int. Cl. C07C 1/30; B01J 29/46; B01J 29/40

(52) U.S. Cl. CPC C07C 1/30; B01J 29/405; B01J 29/40; B01J 29/46; B01J 2229/20; C07C 2529/40; C07C 2529/46

(57) ABSTRACT

Disclosed is a method for converting an alkyl halide to an olefin. The method can include contacting a zeolite catalyst having a chemical formula of M_{y/n}H_{(x-y)}AlₓSi₍₉₆₋ₓ₎O₁₉₂, where M is a metal cation having a valence of n under reaction conditions sufficient to produce an olefin hydrocarbon product comprising C₂ to C₄ olefins. M can include cations of metals from Groups IA, IIA, IIIA. IVB, VB, VIB VIIB, IB, IIB, IIIA or IVA, or any combination of metal cations thereof and y is 0.4≦y≦5.0.

### CROSS REFERENCE TO RELATED APPLICATIONS

This application claims the benefit of priority of U.S. Provisional Patent Application No. 62/167,030, filed May 27, 2015, which is hereby incorporated by reference in its entirety.

### BACKGROUND OF THE INVENTION

### A. Field of the Invention

The invention generally concerns the use of cation exchanged ZSM-5 zeolites as catalysts in the production of C₂ to C₄ olefins from alkyl halides. In particular, the zeolite catalyst can have a MFI structure with a general formula of M_{y/n}H_{(x-y)}AlₓSi₍₉₆₋ₓ₎O₁₉₂ where M is the cation of valence n, with a high selectivity for propylene production and stable catalyst performance over prolonged periods of use.

### B. Description of Related Art

Descriptions of units, abbreviation, terminology, etc. used throughout the present invention are listed in Table 1.

Light olefins such as ethylene and propylene are used by the petrochemical industry to produce a variety of key chemicals that are then used to make numerous downstream products. By way of example, both of these olefins are used to make a multitude of plastic products that are incorporated into many articles and goods of manufacture. FIG. 1 is a chart that provides non-limiting uses of propylene. Currently, the main process used to prepare light olefins is via steam cracking of naphtha. This process, however, requires high temperature and large amount of naphtha which, in-turn, is obtained from the distillation of crude oil. While this process is viable, its reliance on crude oil can be a rate-limiting step, and can increase the manufacturing costs associated with ethylene and propylene production.

Methane activation to higher hydrocarbons, specifically to light olefins, has been the subject of great interest over many decades. Recently, the conversion of methane to light olefins via a two-step process that includes conversion of methane to methyl halide, particularly to methyl mono-halide, for example, to methyl chloride, followed by conversion of the halide to light olefins has attracted great attention. Typically, zeolite (e.g., ZSM-5) or zeolite type catalysts (e.g., SAPO-34) have been tried for the methyl chloride (or other methyl halide) conversion. However, the selectivity to a desired olefin (e.g., propylene) and the rapid catalyst deactivation for the halide reaction remain the major challenges for commercial success.

One of the most commonly used catalysts in petrochemical industry is ZSM-5 zeolite. It is a medium pore zeolite with pore size about 5.5 Å and is shown to convert methyl halide, particularly methyl chloride or methyl bromide, to C₂-C₄ olefins and aromatics under methyl halide reaction conditions. Whereas molecular sieve SAPO-34, an iso-structure of chabazite zeolite having small pore openings (3.8 Å), is shown to convert methyl halide to ethylene and propylene and small amounts of C₄ olefins. Both catalysts, however, are shown to deactivate rapidly during methyl halide conversion due to carbon deposition on the catalysts.

Recently, an attempt has been made to produce propylene from methyl chloride and methyl bromide (See, Xu et al. in Fluoride-treated HZSM-5 as a highly selective stable catalyst for the production of propylene from methyl halides, Journal of Catalysis, 2012, vol. 295, pp. 232-241). Xu et al. treated a ZSM-5 catalyst with fluoride to increase both the propylene selectivity and stability of the catalyst. Notably, however, the collaborators observed that untreated HZSM-5 catalysts showed considerable catalyst deactivation. Such deactivation of the catalyst requires frequent or continuous catalyst regeneration or frequent catalyst change-out resulting in inefficient plant operation or in the use of more catalysts to produce the desired amounts of ethylene and propylene, which increase the manufacturing costs. Still further, the catalytic material has to be re-supplied in shorter time intervals, which oftentimes requires the reaction process to be shut down.

TABLE 1

| Abbreviation | Description |
|---|---|
| Å | Angstrom |
| ° C. | degree Celsius |
| cm³/min | cubic centimeter per min |
| G | Gram |
| H | Hour |
| m²/g | meter square per gram |
| mole % | mole percent |
| % | Percent |
| psig | pound per square inch gauge |
| WHSV | weight hourly space velocity |
| Wt. % | weight percent |

### SUMMARY OF THE INVENTION

A discovery has been made that solves the problems associated with low molecular weight olefin production. In particular, the discovery is premised on the use of cation exchanged ZSM-5 zeolites as catalysts to convert alkyl halides to C₂-C₄ olefins. The catalysts of the present invention have shown increased selectivity towards propylene and butylene production as well as catalyst performance stability during prolonged periods of use. The decline of catalytic activity with time on stream slows down, thereby allowing for the continued use of the catalysts over longer period of time. Without wishing to be bound by theory, it is believed that the partial cation-exchange of ZSM-5 resulting in MHZSM-5 where M is cations of a metal from Groups IA, IIA, IIIB, IVB, VB, VIB VIIB, VIIIB, IB, IIB, IIIA, or IVA (Columns 1-14) of the Periodic Table, or any combination of cations thereof, provides both an increased selectivity for the production of propylene and butylene from alkyl halides and improved stability of catalyst performance over prolonged periods of use us.

In one aspect of the present, a method for converting an alkyl halide to an olefin is described. The method can include contacting a zeolite catalyst having a MFI structure with a chemical composition as shown in Formula (I) with a feed comprising an alkyl halide under reaction conditions sufficient to produce an olefin hydrocarbon product comprising C₂ to C₄ olefins.

M_{y/n}H_{(x-y)}AlₓSi₍₉₆₋ₓ₎O₁₉₂ (I)

where M is a metal cation of Group IA, IIA, IIIB. IVB, VB, VIB VIIB, VIIIB, IB, IIB, IIIA, IVA, or any combination of cations thereof, and n is the valence of the charge balancing cation M. The x value can be varied to obtain any of the desired SARs discussed throughout the specification and claims, and y can range from 0.4≦y≦5.0.

In some aspects of the invention, the zeolite catalyst can include protons H⁺ in addition to the metal cation. The metal cation M can be magnesium (Mg), calcium (Ca), strontium (Sr), cobalt (Co), copper (Cu), zinc (Zn), gallium (Ga), or any combination thereof. In some instances, the metal cation is a combination of Mg²⁺ and Ga³⁺ ions. In some instances, the metal cation is Mg²⁺ and the zeolite catalyst can include 41 to 47 wt. % silicon (Si), 0.20 to 3.6 wt. % aluminum (Al), and 0.05 to 0.25 wt. % Mg. In some aspects, the metal cation is Ca²⁺ and the zeolite catalyst can include 41 to 47 wt. % silicon (Si), 0.20 to 3.6 wt. % Al, and 0.08 to 0.12 wt. % Ca. In other embodiments, the metal cation is Sr²⁺ and the zeolite catalyst can include 41 to 47 wt. % silicon (Si), 0.20 to 3.6 wt. % Al, and 0.23 to 0.27 wt. % Sr. The zeolite of the catalyst of the present invention can have a silica to alumina ratio (SAR) of 250 to 300, 270 to 290, 275 to 285, or around 278, or from 30 to 200.

At reaction conditions, including a temperature of greater than 300° C., preferably a temperature of 400 to 500° C., a weight hourly space velocity (WHSV) of greater than 0.5 h⁻¹, preferably 2.0 to 3.5 h⁻¹, and a pressure of less than 10 psig, preferably less than 5 psig, the zeolite catalyst can provide alkyl halide conversion of at least 30%, a combined propylene and butylene selectivity of at least 40%, and a C₂ to C₄ olefin combined selectivity of at least 50%. In certain instances, the propylene selectivity is about 30% to 60% and the butylene selectivity is about 10 to 20%. In some instances, the C₂ to C₄ olefins selectivity is 50 to 80% at 30 to 80% alkyl halide conversion. In particular aspects, the feed can include about 10 to 30 mole %, or 20 mole % of the alkyl halide such as a methyl halide. Non-limiting examples of methyl halides include methyl chloride, methyl bromide, methyl fluoride, or methyl iodide, or any combination thereof. In particular embodiments, the alkyl halide is methyl chloride. In one aspect, the catalyst has not been subjected to a halide treatment. In particular aspects, the zeolite catalyst has not been treated with phosphorus or a halide (e.g., it has not been subjected to fluoride treatment) or deposited with metal (e.g., Pt, Pd, etc.).

The method can further include collecting or storing the produced olefin hydrocarbon product along with using the produced olefin hydrocarbon to produce a petrochemical or a polymer. Additionally, the used and deactivated zeolite catalyst can be regenerated.

The decrease of alkyl halide conversion can be attributed to carbon deposition on the zeolite catalyst. The carbon deposition causes the blockage of active sites resulting in decrease of conversion. The spent catalyst can be regenerated by burning the deposited carbon. Such carbon burning can generally be performed by heating the spent catalyst in oxygen, preferably diluted oxygen, often used air, at temperature between 400 to 600° C.

In another aspect of the present invention there is disclosed a zeolite catalyst capable of converting a feed that includes an alkyl halide to an olefin hydrocarbon product that includes C₂ to C₄ olefins. The zeolite catalyst can include the catalyst having a chemical composition as shown in Formula (I) and an alkyl halide conversion of at least 30%, a combined propylene and butylene selectivity of at least 40%, and a C₂ to C₄ olefin combined selectivity of at least 50% at reaction conditions including a temperature of greater than 300° C., preferably temperature of 400 to 500° C., a weight hourly space velocity (WHSV) of greater than 0.5 h⁻¹, preferably, 1.0 to 10.0 h⁻¹, more preferably 1.5 to 5.0 h⁻¹, and a pressure of less than 10 psig, preferably less than 5 psig. The catalyst can include M cations from Groups IIA or Group IIIA such as magnesium and gallium or both. In some instances, the M cation can be Mg, Ca, Sr, Co, Cu, Zn, or Ga cations, or any combination thereof. In some instances, the M cation is Mg²⁺ and the zeolite catalyst can include 41 to 47 wt. % silicon (Si), 0.20 to 3.6 wt. % aluminum (Al), and 0.05 to 0.25 wt. % Mg. In some aspects, the M cation is Ca²⁺ and the zeolite catalyst can include 41 to 47 wt. % Si, 0.22 to 3.6 wt. % Al, and 0.08 to 0.12 wt. % Ca. In other embodiments, the M cation is Sr²⁺ and the zeolite catalyst can include 41 to 47 wt. % Si, 0.27 to 3.6 wt. % Al, and 0.23 to 0.27 wt. % Sr. The zeolite of the catalyst of the present invention can have a silica to alumina ratio (SAR) of 30 to 100, or 30 to 250, or 100 to 300, or 200 to 400.

In still another embodiment of the present invention there is disclosed a system for producing olefins. The system can include an inlet for a feed that includes the alkyl halide discussed above and throughout this specification, a reaction zone that is configured to be in fluid communication with the inlet, and an outlet configured to be in fluid communication with the reaction zone to remove an olefin hydrocarbon product from the reaction zone. The reaction zone can include any one of the zeolite catalysts discussed above and throughout this specification. During use, the reaction zone can further include the alkyl halide feed and the olefin hydrocarbon product (e.g., ethylene, propylene, or butylene, or a combination thereof). The temperature of the reaction zone can be 325 to 450° C. The system can include a collection device that is capable of collecting the olefin hydrocarbon product.

In another aspect of the present invention there is disclosed embodiments 1 to 44. Embodiment 1 is a method for converting an alkyl halide to an olefin, the method comprising contacting a zeolite catalyst having a MFI structure with a chemical formula:

M_{y/n}H_{(x-y)}AlₓSi₍₉₆₋ₓ₎O₁₉₂

with a feed comprising an alkyl halide under reaction conditions sufficient to produce an olefin hydrocarbon product comprising C₂ to C₄ olefins, where M is a metal cation of group IA, IIA, IIIB. IVB, VB, VIB VIIB, VIIIB, IB, IIB, IIIA or IVA, or any combination of cations thereof, and n is the valence of the charge balancing cation M, and y is 0.4≦y≦5.0, and where the zeolite catalyst optionally contains H⁻ in addition to the metal cation. Embodiment 2 is the method of embodiment 1, wherein the zeolite catalyst has a higher alkyl halide conversion to an olefin when compared with H/ZSM-5. Embodiment 3 is the method of any one of embodiments 1 to 2, wherein M is a Group IIA or Group IIIA metal cation or any combination thereof. Embodiment 4 is the method of embodiment 3, wherein M is magnesium (Mg). Embodiment 5 is the method of embodiment 4, wherein the catalyst comprises 46 to 47 wt. % silicon (Si), 0.20 to 0.30 wt. % aluminum (Al), and 0.05 to 0.25 wt. % Mg. Embodiment 6 is the method of embodiment 3, wherein M is calcium (Ca). Embodiment 7 is the method of embodiment 6, wherein the catalyst comprises 41 to 47 wt. % Si, 0.22 to 3.6 wt. % Al, and 0.08 to 0.12 wt. % Ca. Embodiment 8 is the method of embodiment 3, wherein M is Strontium (Sr). Embodiment is the method of embodiment 8, wherein the catalyst comprises 46 to 47 wt. % Si, 0.27 to 0.31 wt. % Al, and 0.23 to 0.27 wt. % Sr. Embodiment 10 is the method of embodiment 1, wherein M is magnesium (Mg), calcium (Ca), Strontium (Sr), cobalt (Co), copper (Cu), zinc (Zn), or gallium (Ga), or any combination thereof. Embodiment 11 is the method of embodiment 10, wherein M is a combination of Mg and Ga. Embodiment 12 is the method of any one of embodiments 1 to 11, wherein the reaction conditions include a temperature of greater than 300° C., a weight hourly space velocity (WHSV) of greater than 0.5 h⁻¹, and a pressure of less than 5 psig, or preferably a temperature of 400 to 500° C., a weight hourly space velocity (WHSV) of 1.0 to 5.0 h⁻¹, and a pressure of less than 5 psig. Embodiment 13 is the method of any one of embodiments 1 to 12, wherein propylene selectivity is 30% to 60% and butylene selectivity is 10 to 20%. Embodiment 14 is the method of any one of embodiments 1 to 13, wherein the zeolite catalyst has an alkyl halide conversion of at least 30%, at least 30 to 80%, or at least 40 to 80%. Embodiment 15 is the method of any one of embodiments 1 to 14, wherein the combined selectivity of the C₂ to C₄ olefins is 50 to 80% at 30 to 80% alkyl halide conversion. Embodiment 16 is the method of any one of embodiments 1 to 15, wherein the silica to alumina ratio (SAR) of the zeolite of the catalyst is 250 to 300, 270 to 290, 275 to 285, or around 278. Embodiment 17 is the method of any one of embodiments 1 to 16, wherein the silica to alumina ratio (SAR) of the zeolite of the catalyst is greater than 30 but less than 500. Embodiment 18 is the method of any one of embodiments 1 to 17, wherein the alkyl halide is a methyl halide. Embodiment 19 is the method of embodiment 18, wherein the feed comprises about 10 mole % or more of the methyl halide. Embodiment 20 is the method of any one of embodiments 18 to 19, wherein the methyl halide is methyl chloride, methyl bromide, methyl fluoride, or methyl iodide, or any combination thereof. Embodiment 21 is the method of any one of embodiments 1 to 20, wherein the catalyst has not been subjected to a halide treatment. Embodiment 22 is the method of any one of embodiments 1 to 21, further comprising collecting or storing the produced olefin hydrocarbon product. Embodiment 23 is the method of any one of embodiments 1 to 22, further comprising using the produced olefin hydrocarbon product to produce a petrochemical or a polymer. Embodiment 24 is the method of any one of embodiments 1 to 23, further comprising regenerating the used zeolite catalyst after at least 40 hours of use.

Embodiment 25 is a zeolite catalyst capable of converting a feed comprising an alkyl halide to an olefin hydrocarbon product comprising C₂ to C₄ olefins, the zeolite catalyst having a MFI structure with a chemical formula:

M_{y/n}H_{(x-y)}AlₓSi₍₉₆₋ₓ₎O₁₉₂

where M is a metal cation of group IA, IIA, IIIA. IVB, VB, VIB VIIB, IB, IIB, IIIA or IVA, or any combination of cations thereof, and n is the valence of the charge balancing cation M, and y is 0.4≦y≦5.0, and where the zeolite catalyst optionally contains H⁺ in addition to the metal cation. Embodiment 26 is the zeolite catalyst of embodiment 25, wherein M is a Group IIA or Group IIIA metal cation or any combination thereof. Embodiment 27 is the zeolite catalyst of embodiment 26, wherein M is Mg. Embodiment 28 is the zeolite catalyst of embodiment 27, wherein the catalyst comprises 41 to 47 wt. % Si, 0.20 to 3.6 wt. % Al, and 0.05 to 0.25 wt. % Mg. Embodiment 29 is the zeolite catalyst of embodiment 28, wherein M is Ca. Embodiment 30 is the zeolite catalyst of embodiment 29, wherein the catalyst comprises 46 to 47 wt. % Si, 0.22 to 0.26 wt. % Al, and 0.08 to 0.12 wt. % Ca. Embodiment 31 is the zeolite catalyst of embodiment 26, wherein M is Sr. Embodiment 32 is the zeolite catalyst of embodiment 31, wherein the catalyst comprises 46 to 47 wt. % Si, 0.27 to 0.31 wt. % Al, and 0.23 to 0.27 wt. % Sr. Embodiment 33 is the zeolite catalyst of embodiment 26, wherein M is Mg, Ca, Sr, Co, Cu, Zn, or Ga, or any combination thereof. Embodiment 34 is the zeolite catalyst of embodiment 33, wherein M is a combination of Mg and Ga. Embodiment 35 is the zeolite catalyst of any one of embodiments 25 to 34, wherein the silica to alumina ratio (SAR) of the zeolite of the catalyst is greater than 30 but less than 500, preferably between 30 and 380. Embodiment 36 is the zeolite catalyst of any one of embodiments 25 to 35, wherein the silica to alumina ratio (SAR) of the zeolite of the catalyst is 30 to 100. Embodiment 37 is the zeolite catalyst of any one of embodiments 25 to 35, wherein the silica to alumina ratio (SAR) of the zeolite of the catalyst is 100 to 400. Embodiment 38 is the zeolite catalyst of any one of claims 25 to 37, wherein the zeolite catalyst has an alkyl halide conversion of at least 30%, at least 30 to 80%, or at least 40 to 80% during use. Embodiment 39 is the zeolite catalyst of any one of embodiments 25 to 38, wherein the selectivity of the C₂ to C₄ olefins is 50 to 80% at 30 to 80% alkyl halide conversion during use. Embodiment 40 is the zeolite catalyst of any one of embodiments 25 to 39, wherein the catalyst has not been subjected to a halide treatment.

Embodiment 41 a system for producing olefins, the system comprising: an inlet for a feed comprising an alkyl halide; a reaction zone that is configured to be in fluid communication with the inlet, wherein the reaction zone comprises any one of the zeolite catalysts of embodiments 25 to 40; and an outlet configured to be in fluid communication with the reaction zone to remove an olefin hydrocarbon product from the reaction zone. Embodiment 42 is the system of embodiment 41, wherein the reaction zone further comprises the feed and the olefin hydrocarbon product. Embodiment 43 is the system of embodiment 42, wherein the olefin hydrocarbon product comprises ethylene, propylene, and butylene. Embodiment 44 is the system of any one of embodiments 41 to 43, further comprising a collection device that is capable of collecting the olefin hydrocarbon product.

The “Periodic Table” as used throughout this Specification is the 2005 Periodic Table published by the Chemical Abstracts Society. Groups IA, IIA, IIIB, IVB, VB, VIB VIIB, VIIIB, IB, IIB, IIIA or IVA used in this Specification correspond to Columns 1-7, 9, and 11-14 respectively of the 2013 IUPAC Periodic Table.

The term “about” or “approximately” is defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

The catalysts of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the catalysts of the present invention are their ability to selectively produce an olefin, and in particular, propylene and butylene, in high amounts, while also remaining stable in terms of activity after prolonged periods of use (e.g., 20 hours and longer).

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1 is a chart listing various chemicals and products that can be made from propylene.

FIG. 2 is a schematic of an embodiment of a system for producing olefins from alkyl halides.

FIG. 3 is a graph of methyl chloride conversion in percent vs time in hours on stream for catalysts of the present invention and comparative catalysts.

FIG. 4 is a graph of selectivity to C₂-C₄ olefins in percent versus time in hours on stream for catalysts of the present invention and a comparative catalyst.

FIG. 5 is a graph of methyl chloride conversion in percent versus time in hours on stream for double ion-exchanged catalysts of the present invention and a comparative catalyst.

FIG. 6 is a graph of selectivity to C₂-C₄ olefins in percent versus time in hours on stream versus for double-ion exchange catalysts of the present invention and a comparative catalyst.

### DETAILED DESCRIPTION OF THE INVENTION

In the petrochemical industry, the principal source for light olefins, such as ethylene and propylene, is steam cracking of the hydrocarbon feed, for example naphtha, LPG or ethane. Using other feedstock, such as methane, has been an attractive alternative to convert the methane to light olefins via a two-step process which consists of conversion of methane to methyl halide, particularly to methyl mono-halide, for example, to methyl chloride followed by conversion of the halide to C₂-C₄ olefins. This alternative, however, has drawbacks. Namely, zeolite (e.g., ZSM-5) and zeolite type catalysts (e.g., SAPO-34) have been utilized as catalysts for the methyl chloride conversion, but low selectivity to a desired olefin (e.g., propylene) and rapid catalyst deactivation in the alkyl halide reaction remain the major challenge to such a process.

A discovery has been made with ZSM-5 zeolite catalysts that alleviates these problems. In particular, the use of zeolites having a MFI structure with a chemical composition as shown in formula (I) and having a SAR of at least 30, but less than 500, surprisingly results in an increase of propylene and butylene production from alkyl halides. The MFI zeolite catalyst can have a composition of M_{y/n}H_{(x-y)}AlₓSi₍₉₆₋ₓ₎O₁₉₂ with cations of metals from Groups IA, IIA, IIIB. IVB, VB VIB VIIB, VIIIB, IB, IIB, IIIA, IVA, or any combination of cations thereof, preferably M is Mg, Ca, Sr, Co, Cu, Zn, or Ga, more preferably, Mg, Ca, Sr, or Ga, and most preferably Mg or MgGa. The catalysts of the present invention display substantially better stability in terms of activity giving an increased alkyl halide conversion of at least 30% with no change in reaction conditions. For example, the catalysts can convert at least about 40 grams of alkyl halide per gram of catalyst while maintaining greater than 30% alkyl halide conversion with no change in reaction conditions. This allows for a more targeted and continuous production of the olefins without having to constantly provide additional catalyst to the reaction process.

These and other non-limiting aspects of the present invention are discussed in further detail in the following sections.

### A. Cation Exchanged ZSM-5 Catalysts

ZSM-5 zeolite is a porous material containing intersecting two-dimensional pore structure with 10-membered ring openings. This zeolite and its preparation are described in U.S. Pat. No. 3,702,886, which is herein incorporated by reference. In the present invention, the ZSM-5 zeolite may include those having a silica to alumina (SiO₂/Al₂O₃) ratio (SAR) of at least about 30, but less than 500, or 25 to 300, 60 to 290, 275 to 285, or around or 278. The x variable in Formula 1 can be varied to achieve any of these SARs. In the other aspects of the present invention, the ZSM-5 zeolite may include those having a silica to alumina (SiO₂/Al₂O₃) ratio (SAR) of about 32 or 58. Modified and unmodified ZSM-5 zeolites are commercially available from a wide range of sources, e.g. Zeolyst International (Valley Forge, Pa., USA), Clariant International Ltd. (Munich, Germany), Tricat Inc. (McAlester, Okla., USA). In preferred embodiments, unmodified HZSM-5 is used, which is commercially available from at least the aforementioned sources. However, modified ZSM-5 as well as other zeolites such as ZSM-11, ZSM-23, silicalite, ferrierite, and mordenite can also be used. While the SAR for each of the zeolites can vary, in preferred aspects, a SAR of at least 30, at least 50, or at least 200 for the additional zeolites is preferred. In certain aspects, the ZSM-5 zeolite catalyst of the present invention is acidic H-form and can be synthesized or commercially obtained.

The ZSM-5 zeolite using known ion exchange processes can be ion exchanged again with the desired cation of metal from Groups IA, IIA, IIIB. IVB, VB VIB VIIB, VIIIB, IB, IIB, IIIA, IVA using known ion exchange processes or the processes described throughout the specification (See, for example, catalyst preparation in Example 1). In one instance, the ZSM-5 zeolite can be combined with an ion exchange solution containing the metal cation at a desired pH, for example, with a magnesium acetate or magnesium nitrate solution at a pH of about 6 to 8 for a desired amount of time. The ion exchanged catalyst can be removed from the solution by filtration, dried, and then calcined at temperatures from about 250° C. to about 750° C., preferably from about 350° C. to about 550° C., and more preferably from about 400° C. to about 450° C. to produce a catalyst having the chemical composition shown in Formula I. In some embodiments, the ZSM-5 catalyst with composition M_{y/n}H_{(x-y)}AlₓSi₍₉₆₋ₓ₎O₁₉₂ can be ion exchanged again with the desired cation of metal from Groups IA, IIA, IIIB. IVB, VB VIB VIIB, VIIIB, IB, IIB, IIIA, IVA using known ion exchange processes or the processes described throughout the specification (See, for example, catalyst preparation in Example 1).

### B. Alkyl Halide Feed

The alkyl halide feed can include one or more alkyl halides. The alkyl halide feed may contain alkyl mono halides, alkyl dihalides, alkyl trihalides, preferably alkyl mono halide with less than 10% of other halides relative to the total halides. The alkyl halide feed may also contain nitrogen, helium, steam, and so on as inert diluent compounds. The alkyl halide in the feed may have the following structure: CₙH₍₂ₙ₊₂₎₋ₘXₘ, where n and m are integers, n ranges from 1 to 5, preferably 1 to 3, even more preferably 1, m ranges 1 to 3, preferably 1, X is Br, F, I, or Cl. Non-limiting examples of alkyl halides include methyl chloride, methyl bromide, methyl fluoride, or methyl iodide, or any combination thereof. In particular aspects, the feed may include about 10, 15, 20, 40, 50 mole % or more of the alkyl halide. In particular embodiments, the feed contains up to 20 mole % of an alkyl halide. In preferred aspects, the alkyl halide is methyl chloride. In a particular embodiment, the alkyl halide is methyl chloride or methyl bromide.

The alkyl halide, particularly methyl chloride CH₃Cl (see Equation 2 below), is commercially produced by thermal chlorination of methane at a temperature of 400° C. to 450° C. and a raised pressure. Catalytic oxychlorination of methane to methyl chloride is also known. In addition, methyl chloride is industrially made by reaction of methanol and HCl at 180° C. to 200° C. using a catalyst. Alternatively, methyl halides are commercially available from a wide range of sources, such as Praxair (Danbury, Conn., USA) Sigma-Aldrich® Co. LLC, (St. Louis, Mo., USA), BOC Sciences USA (Shirley, N.Y., USA). In preferred aspects, methyl chloride and methyl bromide can be used alone or in combination.

### C. Olefin Production

The ion-exchanged ZSM-5 zeolites of the present invention catalyze the conversion of alkyl halides to light olefins such as ethylene, propylene and butylenes. The following non-limiting two-step process is an example of conversion of methane to methyl chloride and conversion of methyl chloride to ethylene, propylene and butylene. The second step illustrates the reactions that are believed to occur in the context of the present invention:

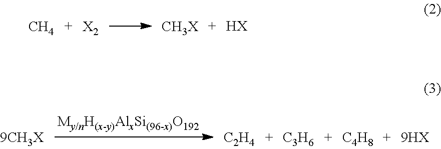

where X is Br, F, I, or Cl, and non-limiting examples of M include Mg, Ca, Sr, Co, Cu, Zn, or Ga, or any combination thereof having a valence of n. Besides the C₂-C₄ olefins the reaction may produce other hydrocarbons such as methane, C₅ olefins, C₂-C₅ alkanes and aromatic compounds such as benzene, toluene and xylene.

Conditions sufficient for olefin production (e.g., ethylene, propylene and butylene as shown in Equation 3) include temperature, time, alkyl halide concentration, space velocity, and pressure. The temperature range for olefin production may range from about 300° C. to 500° C., preferably ranging 350° C. to 450° C. A weight hourly space velocity (WHSV) of alkyl halide higher than 0.5 h⁻¹ can be used, preferably between 1.0 and 10.0 h⁻¹, more preferably between 1.5 and 5.0 h⁻¹. The conversion of alkyl halide is carried out at a pressure less than 5 psig, preferably less than 1 psig, more preferably less than 0.5 psig, or at atmospheric pressure. The conditions for olefin production may be varied based on the type of the reactor.

The reaction can be conducted over the M_{y/n}H_{(x-y)}AlₓSi₍₉₆₋ₓ₎O₁₉₂ catalysts having the particular metal cation for prolonged periods of time without changing or re-supplying fresh catalyst or catalyst regeneration. This is due to the stability or slower deactivation of the catalysts of the present invention. Therefore, the reaction can be performed for a period of time until the level of alkyl halide conversion reaches to a preset level (for example, 30%). In preferred aspects, the reaction can continuously run for 20 hours or 20 to 50 hours or longer without having to stop the reaction to re-supply fresh catalyst or regenerate catalyst. The method can further include collecting or storing the olefin hydrocarbon product along with using the produced olefin hydrocarbon to make a petrochemical or a polymer.

### D. Catalyst Activity/Selectivity

Catalytic activity as measured by alkyl halide conversion can be expressed as the % moles of the alkyl halide converted with respect to the moles of alkyl halide fed. In particular aspects, the combined selectivity of ethylene, propylene and butylene is at least 50% under certain reaction conditions. In certain instances, the selectivity to propylene is about 30% or higher, the selectivity to butylene is about 10% or higher, and ethylene selectivity is about 12% or less. It was surprisingly found that the introduction of magnesium ions Mg²⁺ improved the percent conversion and slowed down deactivation compared to the parent HZ SM-5 catalyst having the same SAR. In some instances, the catalysts of the invention increase activity and selectivity with increasing metal percentage in the zeolite. For example, catalyst having 0.2 wt. % of metal have higher activity and selectivity than the catalysts having 0.05 wt. % of the same metal. In some instances, methyl chloride conversion has less than a 10% of a drop in conversion and in some cases less than 5% of a drop in conversion over ion exchanged ZSM-5 as compared to the parent HZSM-5 catalyst for the same time on stream. In other instances, a double ion-exchanged catalyst (for example, MgGaZSM-5 and GaMgZSM-5 catalysts) can provide a higher conversion of methyl chloride and better selectivity to C₂-C₄ olefins, and deactivate significantly slower compared to the HZSM-5 having the same SAR.

As an example, methyl chloride CH₃Cl is used here to define conversion and selectivity of products by the following formulas:

$$\% {CH}_{3} Cl Conversion = \frac{{( {CH}_{3} Cl )}^{°} - ( {CH}_{3} Cl )}{{( {CH}_{3} Cl )}^{°}} \times 100$$

where, (CH₃Cl)° and (CH₃Cl) are moles of methyl chloride in the feed and reaction product, respectively.

Selectivity defined as C-mole % are determined for ethylene, propylene, and so on as follows:

$$\% Ethylene Selectivity = \frac{2 ( {C}_{2} {H}_{4} )}{{( {CH}_{3} Cl )}^{°} - ( {CH}_{3} Cl )} \times 100$$

where the numerator is the carbon adjusted mole of ethylene and the denominator is the mole of the methyl chloride converted.

Selectivity for propylene can be expressed as:

$$\% Propylene Selectivity = \frac{3 ( {C}_{3} {H}_{6} )}{{( {CH}_{3} Cl )}^{°} - ( {CH}_{3} Cl )} \times 100$$

where the numerator is the carbon adjusted mole of propylene and the denominator is the mole of the methyl chloride converted.

Selectivity for butylene may be expressed as:

$$\% Butylene Selectivity = \frac{4 ( {C}_{4} {H}_{8} )}{{( {CH}_{3} Cl )}^{°} - ( {CH}_{3} Cl )} \times 100$$

where the numerator is the carbon adjusted mole of butylene and the denominator is the mole of the methyl chloride converted.

### E. Olefin Production System

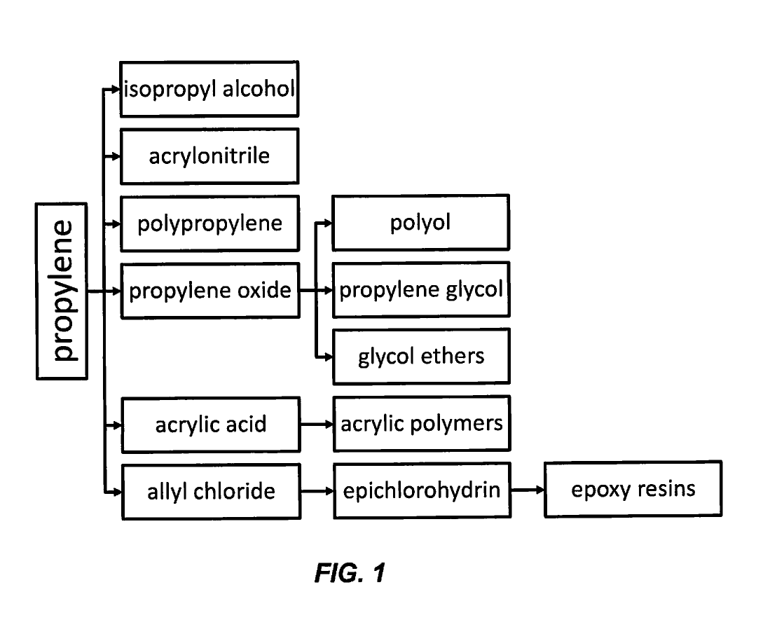

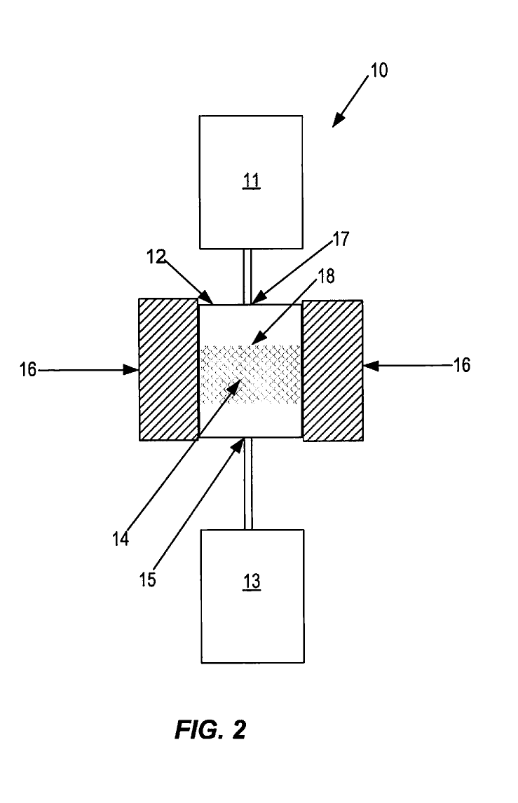

Referring to FIG. 2, a system 10 is illustrated, which can be used to convert alkyl halides to olefin hydrocarbon products with the M_{y/n}H_{(x-y)}AlₓSi₍₉₆₋ₓ₎O₁₉₂ catalysts of the present invention. The system 10 can include an alkyl halide source 11, a reactor 12, and a collection device 13. The alkyl halide source 11 can be configured to be in fluid communication with the reactor 12 via an inlet 17 on the reactor. As explained above, the alkyl halide source can be configured such that it regulates the amount of alkyl halide feed entering the reactor 12. The reactor 12 can include a reaction zone 18 having the M_{y/n}H_{(x-y)}AlₓSi₍₉₆₋ₓ₎O₁₉₂ catalyst 14 of the present invention. The amounts of the alkyl halide feed 11 and the catalyst 14 used can be modified as desired to achieve a given amount of product produced by the system 10. Non-limiting examples of reactors that can be used include fixed-bed reactors, fluidized bed reactors, bubbling bed reactors, slurry reactors, rotating kiln reactors, or any combinations thereof when two or more reactors are used. In preferred aspects, reactor 12 that can be used is a fixed-bed reactor, e.g. a fixed-bed tubular stainless steel coated inside with silica reactor, which can operate at atmospheric pressure. The reactor 12 can include an outlet 15 for products formed in the reaction zone 18. The reaction products can include ethylene, propylene and butylene. The collection device 13 can be in fluid communication with the reactor 12 via the outlet 15. Both the inlet 17 and the outlet 15 can be open and closed as desired. The collection device 13 can be configured to store, further process, or transfer desired reaction products, e.g. C₂-C₄ olefins, for other uses. By way of example only, FIG. 1 provides non-limiting uses of propylene produced from the catalysts and processes of the present invention. Still further, the system 10 can also include a heating source 16. The heating source 16 can be configured to heat the reaction zone 18 to a temperature sufficient (e.g., 325 to 450° C.) to convert the alkyl halides in the alkyl halide feed to olefin hydrocarbon products. A non-limiting example of a heating source 16 can be a temperature controlled furnace. Additionally, any unreacted alkyl halide can be recycled and included in the alkyl halide feed to increase the overall conversion of alkyl halide to olefin products. Further, certain co-products, such as C₅+ olefins and C₂+ alkanes, can be separated and used in other processes to make commercially valuable chemicals, for example propylene. This will increase the efficiency and commercial value of the alkyl halide conversion process of the present invention.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1

(Catalyst of the Invention Preparation)

Catalyst A. 15 grams of the ZSM-5 zeolite with SAR of 32 purchased from Zeolyst International Inc. were immersed in 250 ml of 0.1 molar aqueous solution of magnesium acetate and stirred for 2 hours at room temperature and pH 6.0. The slurry was then filtered and washed with 1 liter of de-ionized water. The precipitate was dried at 120° C. for 3 hours and calcined in air at 450° C. for 10 hours.

Catalyst B. 15 grams of the ZSM-5 zeolite with SAR of 58 obtained from Zeolyst International Inc. was added to 250 ml of 0.1 molar aqueous solution of magnesium acetate solution. The slurry was stirred for 2 hours at room temperature and a pH of 6.0, filtered and washed with 1 liter of distilled water. The precipitate was dried at 120° C. for 3 hours and calcined in air at 450° C. for 10 hours.

Catalyst C. 50 grams of the ZSM-5 zeolite with SAR of 258 purchased from Zeolyst International Inc. were immersed in 0.5 molar aqueous solution of magnesium acetate and stirred for 2 hours at room temperature and a pH 7.7. The slurry was then filtered and washed with 1 liter of de-ionized water. The precipitate was dried at 300° C. for 14 hours and underwent one more ion exchanging procedure with 0.5 molar solution of magnesium acetate. After filtering and washing the precipitate was calcined in air at 300° C. for 14 hours and at 450° C. for 10 hours.

Catalyst D. 50 grams of the ZSM-5 zeolite with SAR of 258 from Zeolyst International Inc. were mixed with 0.2 molar solution of magnesium acetate and stirred for 2 hours at room temperature and a pH of 7.6. Then the slurry was filtered and washed with 1 liter of distilled water. The precipitate was dried in air at 300° C. for 14 hours and calcined at 450° C. for 10 hours.

Catalyst E. 50 grams of ZSM-5 zeolite with SAR of 258 from Zeolyst International Inc. were added to 0.5 molar solution of magnesium acetate. The slurry was stirred for 2 hours at room temperature and a pH of 7.6, filtered and washed with de-ionized water. The precipitate was then dried at 300° C. for 14 hours and calcined at 450° C. for 10 hours in air.

Catalyst F. 50 grams of ZSM-5 zeolite with SAR of 258 from Zeolyst International Inc. were immersed in 500 ml of 0.2 molar solution of magnesium acetate solution and stirred for 2 hours at room temperature and a pH of 7.3. The slurry was separated from liquid by filtering and washed with 1 liter of distilled water. The precipitate was dried at 300° C. for 14 hours and again ion exchanged with 0.2 molar solution of magnesium acetate. After filtration and washing the precipitate was dried at 300° C. for 14 hours and calcined at 450° C. for 10 hours in air.

Catalyst G. 100 grams of ZSM-5 zeolite with SAR of 258 from Zeolyst International Inc. were mixed with 0.1 molar aqueous solution of calcium acetate solution. The slurry was stirred for 2 hours at room temperature and a pH of 7.0 and filtered using a Buchner funnel. The precipitate was washed with de-ionized water and dried at 300° C. for 14 hours. The dried solid material was ion exchanged two times more with 0.1 molar solution of calcium acetate under stirring, filtered, washed, dried at 300° C. and calcined at 450° C. in air.

Catalyst H. 100 grams of ZSM-5 zeolite with SAR of 258 from Zeolyst International Inc. were ion exchanged three times with 0.1 molar aqueous solution of strontium acetate. Each time the slurry was stirred for 2 hours at room temperature and a pH of 7.0, filtered, washed with water, and the precipitate was dried at 300° C. for 14 hours. After all these treatments, the material was calcined in air at 450° C. for 10 hours.

Catalyst I. 25 grams of ZSM-5 zeolite with SAR of 258 from Zeolyst International Inc. were added to 250 ml of 0.1 molar solution of cobalt acetate and stirred for 2 hours at room temperature and a pH of 6.0. The slurry was then filtered and washed with 1 liter of distilled water. The precipitate was dried at 300° C. for 14 hours and calcined at 450° C. for 10 hours in air.

Catalyst J. 25 grams of ZSM-5 zeolite with SAR of 258 from Zeolyst International Inc.) were immersed in 250 ml of 0.1 molar solution of copper acetate. The slurry was stirred for 2 hours at room temperature and a pH of 5.2, filtered in a Buchner funnel and washed with distilled water. The precipitate was then dried at 300° C. for 14 hours and calcined at 450° C. for 10 hours in air.

Catalyst K. 25 grams of ZSM-5 zeolite with SAR of 258 from Zeolyst International Inc. were mixed with 250 ml of 0.1 molar of zinc acetate solution. The slurry obtained was stirred for 2 hours at room temperature and a pH of 6.1, filtered and washed with 1 liter of de-ionized water. The precipitate was then dried at 300° C. for 14 hours and calcined at 450° C. for 10 hours in air.

Catalyst L. 25 grams of ZSM-5 zeolite with SAR of 258 from Zeolyst International Inc. were added to 250 ml of 0.1 molar of gallium nitrate solution and stirred for 2 hours at room temperature and a pH of 2.7. The slurry was then filtered in a Buchner funnel and washed with de-ionized water. The precipitate was dried at 300° C. for 14 hours and calcined at 450° C. for 10 hours in air.

Catalyst M. 9.4 gm of Catalyst L were immersed in 0.2 molar solution of magnesium acetate and stirred for 2 hours at room temperature and a pH of 6.9. The slurry was then filtered in a Buchner funnel and washed with 1 liter of de-ionized water. The precipitate was dried at 300° C. for 14 hours and calcined at 450° C. for 10 hours in air.

Catalyst N. 10 gm of Catalyst F were treated under stirring with 0.2 molar solution of gallium nitrate for 2 hours at room temperature and a pH of 2.7. The slurry was then filtered in a Buchner funnel and washed with distilled water. The precipitate was dried at 300° C. for 14 hours and calcined at 450° C. for 10 hours in air.

The elemental composition in weight percent identified by XRF, surface area in m²/g-catalyst determined by N₂ adsorption using BET method and acidity in mmole/g-catalyst measured by ammonia thermo-desorption technique of the above-prepared catalysts are listed in Table 2.

TABLE 2

| | | Element weight % | | | Surface | Acidity, |
|---|---|---|---|---|---|---|
| Catalyst | Name (SAR) | Si | Al | Metal | area, m²/g | mmole/g |
| A | MgZSM-5 | 43.38 | 2.56 | 0.1 | 355 | 1.05 |
| | (32) | | | | | |
| B | MgZSM-5 | 41.0 | 1.47 | 0.1 | 384 | 0.78 |
| | (58) | | | | | |
| C | MgZSM-5 | 46.68 | 0.25 | 0.05 | 360 | 0.15 |
| | (278) | | | | | |
| D | MgZSM-5 | 45.56 | 0.24 | 0.09 | 375 | 0.18 |
| | (278) | | | | | |
| E | MgZSM-5 | 46.59 | 0.24 | 0.16 | 372 | 0.19 |
| | (278) | | | | | |
| F | MgZSM-5 | 46.35 | 0.24 | 0.22 | 375 | 0.19 |
| | (278) | | | | | |
| G | CaZSM-5 | 46.48 | 0.24 | 0.10 | 370 | 0.12 |
| | (278) | | | | | |
| H | SrZSM-5 | 46.34 | 0.29 | 0.25 | 377 | 0.13 |
| | (278) | | | | | |
| I | CoZSM-5 | 45.3 | 0.28 | 0.06 | 375 | 0.13 |
| | (278) | | | | | |
| J | CuZSM-5 | 45.0 | 0.28 | 0.59 | ND** | 0.26 |
| | (278) | | | | | |
| K | ZnZSM-5 | 46.2 | 0.28 | 0.07 | 377 | 0.12 |
| | (278) | | | | | |
| L | GaZSM-5 | 45.4 | 0.27 | 0.12 | 374 | 0.14 |
| | (278) | | | | | |
| M | MgGaZSM-5 | 46.2 | 0.28 | 0.14* | 378 | 0.15 |
| | (278) | | | | | |
| N | GaMgZSM-5 | 45.3 | 0.28 | 0.23* | 372 | 0.13 |
| | (278) | | | | | |

*Combined weight % of Mg and Ga
**Not Determined

### Example 2

(Comparative Catalyst Preparation)

The comparative HZSM-5 (32) and HZSM-(58) catalysts were obtained from Zeolyst International Inc. These catalysts were used as starting materials in the preparation of comparative catalysts C1 and C2 in the reaction of methyl chloride conversion. The elemental composition in weight percent, surface area in m²/g and acidity in mmole/g of the comparative catalysts C1 and C2 are listed in Table 3.

The comparative HZSM-5 (278) catalyst was prepared by calcining in air at 530° C. for 10 hours the HZSM-5 zeolite powder sample obtained from Zeolyst International Inc. The calcined material was used in the preparation of catalysts C-N and as comparative catalyst C3 for the conversion of methyl chloride to olefins. The elemental composition in weight percent, surface area in m² per g of catalyst and acidity in mmole per g of catalyst for the comparative catalyst C3 are listed in Table 3.

TABLE 3

| | | Element weight % | | Surface | Acidity, |
|---|---|---|---|---|---|
| Catalyst | Name (SAR) | Si | Al | area, m²/g | mmole/g |
| C1 | HZSM-5 (32) | 43.62 | 3.63 | 370 | 0.94 |
| C2 | HZSM-5 (58) | 43.42 | 1.44 | 382 | 0.76 |
| C3 | HZSM-5 (278) | 46.24 | 0.32 | 389 | 0.10 |

### Examples 3-6

(Comparison of Inventive Metal Ion-Exchanged Catalyst A and B to Comparative Catalysts C1 and C2)

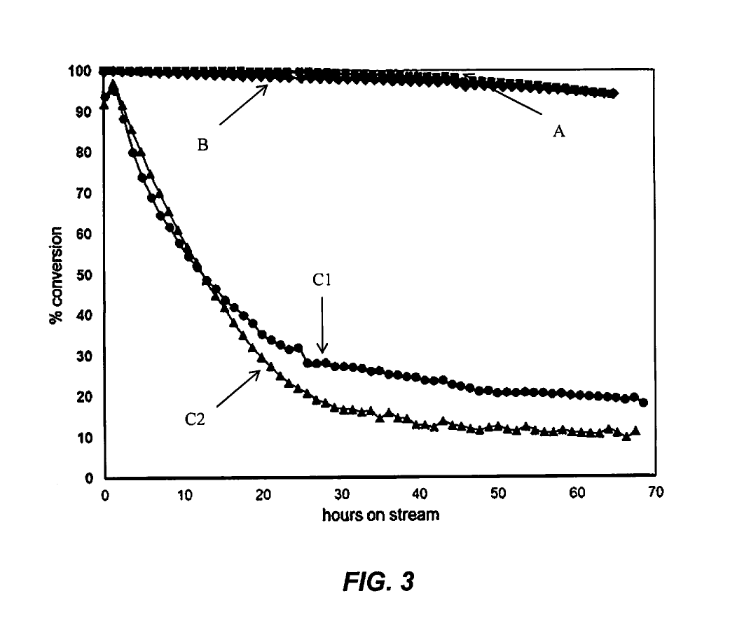

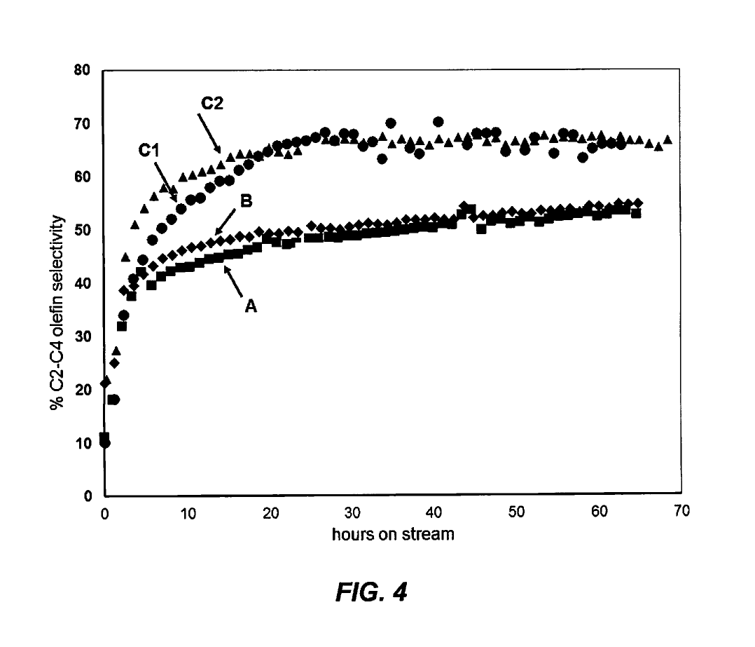

Each of the powder inventive catalysts A and B and comparative catalysts C1 and C3 were first pressed into tablet and then crushed and sieved between 20 and 40 mesh screens. A measured amount of the 20-40 mesh sized catalyst (typically 1.0 g) was loaded in a tubular SS-316½-inch OD reactor. The catalyst was dried under N₂ flow of 100 cm³/min at 200° C. for 1 hour and then the temperature was raised to 300° C. at which N₂ flow was replaced by methyl chloride (20 mole % CH₃Cl in N₂) flowing at a rate of 100 cm³/min. The weight hourly space velocity (WHSV) of CH₃Cl was about 2.8 h⁻¹. The reactor inlet pressure was <5 psig. After an initial period of reaction at 300° C. for about 2 to 3 hours the catalyst bed temperature was increased to about 350° C. The percent methyl chloride conversion and product selectivity after 20 hours on stream for Example 4 are listed in Table 4. FIG. 3 presents a graph of % methyl chloride conversion versus the hours on stream for the tested catalysts. FIG. 4 is a graph of the selectivity to C₂-C₄ olefins as a function of time on stream over catalysts listed in Table 4. Data C1 is comparative catalyst C1, data C2 is comparative catalyst C2, data A is inventive catalyst A, and data B is inventive catalyst B. As is seen in FIG. 3, by the end of the 70-hour nonstop run methyl chloride conversion dropped from about 95% to about 10% on HZSM-5 with SAR 32 and to about 20% on HZSM-5 with SAR 58. Their ion-exchanged forms containing Mg²⁺ cations displayed much better stability to give high methyl chloride conversions of about 95% after 65 hours of continuous testing. During this long run, methyl chloride conversion over both MgZSM-5 catalysts decreased only by 5% from 100 to 95%.

TABLE 4

| | Conversion and Selectivity at | | | | |
|---|---|---|---|---|---|
| | 20 hours on stream (%) | | | | |
| | | | | | C₂-C₄ |
| | | C₂H₄ | C₃H₆ | C₄H₈ | Olefin |
| Catalyst: | CH₃Cl | Selec- | Selec- | Selec- | Selec- |
| Name (SAR) | Convers. | tivity | tivity | tivity | tivity |
| C1: HZSM-5 (32) | 29.7 | 3.6 | 30.8 | 30.3 | 64.7 |
| A: MgZSM-5 (32) | 99.8 | 3.3 | 22.6 | 21.7 | 47.6 |
| C2: HZSM-5 (58) | 35.1 | 3.2 | 35.0 | 27.2 | 65.4 |
| B: MgZSM-5 (58) | 98.6 | 2.5 | 24.9 | 21.9 | 49.3 |

The data presented in Table 4 and FIG. 3 illustrate that all of the catalysts catalyzed methyl chloride transformation to olefins. Ion-exchanged zeolites containing Mg²⁺ ions were more stable in terms of methyl chloride overall conversion than their parent HZSM-5 zeolites. The data presented in Table 4 and FIG. 4 show also that both MgZSM-5 samples were less selective to olefins than their parent zeolites at a given time on stream but not at a constant conversion. For example, at time on stream of 20 hours combined selectivities to C₂-C₄ olefins over MgZSM-5 with SAR of 32 and MgZSM-5 with SAR of 58 were respectively 47.6% and 49.3%. By comparison, HZSM-5 zeolites amounted to 64.7% C₂-C₄ olefin selectivity.

### Examples 7-17

(Comparison of Inventive Metal Ion-Exchanged Catalysts C-L to Comparative Catalyst C3)

Each of the powder inventive catalysts C-L and comparative catalyst C3 were first pressed into tablet and then crushed and sieved between 20 and 40 mesh screens. A measured amount of the 20-40 mesh sized catalysts (typically 1.0 g) were loaded in a tubular SS-316½-inch OD reactor. The catalyst was dried under N₂ flow of 100 cm³/min at 400° C. for 1 h and then temperature was raised to 450° C. when N₂ flow was replaced by methyl chloride CH₃Cl (20 mole %, balance N₂) flowing at the rate 100 cm³/min. The weight hourly space velocity (WHSV) of CH₃Cl was about 2.8 h⁻¹. The reactor inlet pressure was less than 5 psig. The percent methyl chloride conversion and product selectivity after 3 hours of reaction for Examples 7-17 are presented in Table 5.

TABLE 5

| | Conversion and Selectivity at | | | | |
|---|---|---|---|---|---|
| | 3 hours on stream, % | | | | |
| | | | | | C₂-C₄- |
| | CH₃Cl | C₂H₄ | C₃H₆ | C₄H₈ | Olefins |
| Catalyst: | Conver- | Selec- | Selec- | Selec- | Selec- |
| Name (SAR) | sion | tivity | tivity | tivity | tivity |
| C3: HZSM-5 (278) | 31.2 | 3.6 | 30.0 | 9.2 | 48.8 |
| C: MgZSM-5 (278) | 66.4 | 5.4 | 41.8 | 16.5 | 63.7 |
| D: MgZSM-5 (278) | 69.3 | 6.5 | 49.2 | 15.7 | 71.4 |
| E: MgZSM-5 (278) | 76.8 | 6.1 | 50.3 | 16.8 | 73.2 |
| F: MgZSM-5 (278) | 77.6 | 6.2 | 51.2 | 16.8 | 74.2 |
| G: CaZSM-5 (278) | 46.6 | 5.9 | 43.2 | 13.3 | 62.4 |
| H: SrZSM-5 (278) | 30.8 | 7.0 | 36.7 | 11.2 | 54.9 |
| I: CoZSM-5 (278) | 36.0 | 10.2 | 39.3 | 11.8 | 61.3 |
| J: CuZSM-5 (278) | 48.7 | 11.2 | 32.6 | 10.4 | 54.2 |
| K: ZnZSM-5 (278) | 42.0 | 7.6 | 45.6 | 13.4 | 66.6 |
| L: GaZSM-5 (278) | 38.3 | 8.1 | 40.2 | 11.9 | 60.2 |

The HZSM-5 zeolite with SAR of 278 deactivated rapidly with increasing time on stream of the methyl chloride reaction. For instance, during the 3 hour run chloromethane conversion dropped from 44.9 to 31.2%. Many ion-exchanged forms, such as MgZSM-5, CaZSM-5, CoZSM-5, CuZSM-5, ZnZSM-5 and GaZSM-5, displayed under the same reaction conditions significant better stability to give higher methyl chloride conversion and selectivity to C₂-C₄ olefins compared to the parent HZSM-5. MgZSM-5 zeolites were the most effective and stable catalysts the activity and selectivity of which increased with increasing Mg percentage in the zeolite (see Table 5). Particular MgZSM-5 sample containing 0.22% Mg (catalyst F) displayed at time on stream of ˜3 hours 77.6% conversion of methyl chloride and 74.2% selectivity to C₂-C₄ olefins that were much higher than the 31.2% conversion and 48.8% selectivity obtained on HZSM-5. So the data listed in Table 5 allow conclude that the cation exchanged zeolite catalysts C-L were more active, selective and stable than the comparative catalyst C3 at 450° C., WHSV of 2.8 h⁻¹, and pressure of <5 psig.

### Examples 18-20

(Comparison of Inventive Double Ion-Exchanged Catalyst M and N to Comparative Catalyst C3)

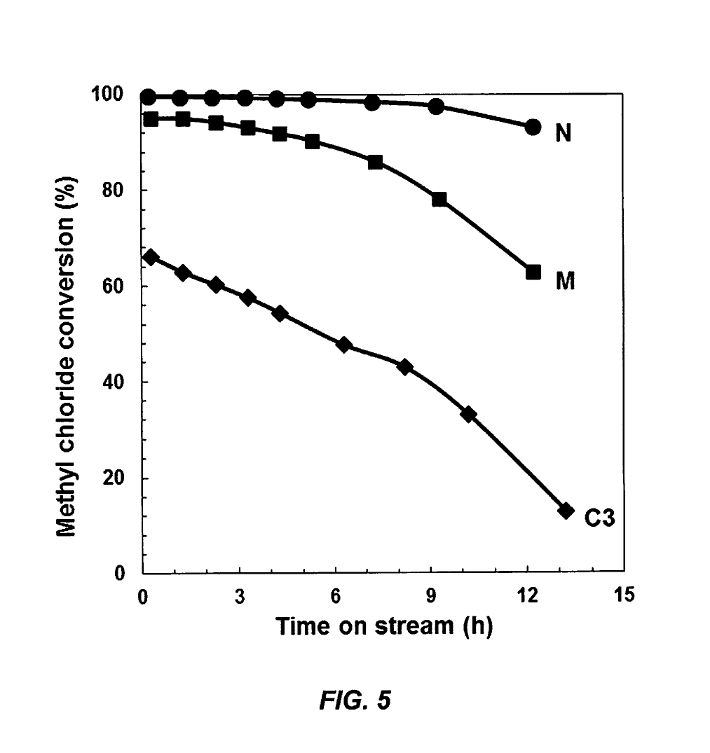

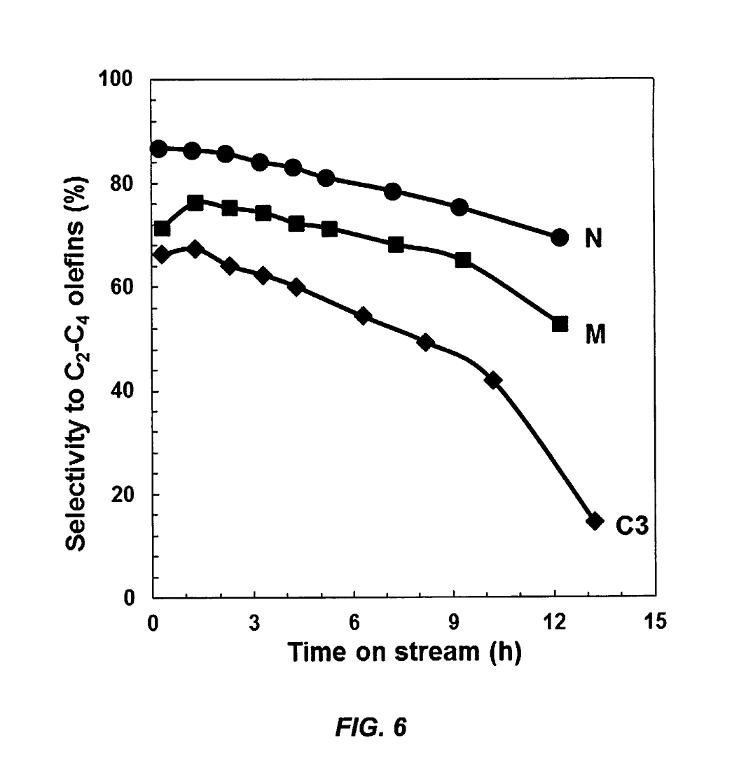

Each of the powder inventive double ion-exchanged catalysts M and N as well as comparative catalyst C3 were first pressed into tablet and then crushed and sieved between 20 and 40 mesh screens. A measured amount of the 20-40 mesh sized catalysts (typically 2.0 g) was loaded in a tubular SS-316½-inch OD reactor. The catalyst was dried under N₂ flow of 100 cm³/min at 400° C. for 1 hour and then the temperature was raised to 450° C. when N₂ flow was replaced by methyl chloride (20 mole % CH₃Cl in N₂) flowing at a rate of 100 cm³/min. The weight hourly space velocity (WHSV) of CH₃Cl was about 1.35 h⁻¹. The reactor inlet pressure was less than 5 psig. The percent methyl chloride conversion and light olefin selectivity after 3 hours on stream for Examples 18-20 are presented in Table 6. FIGS. 5 and 6 show how conversion and selectivity over inventive catalysts M and N, and comparative catalyst C3 changed with time on stream during the 13-hour non-stop run. FIG. 5 is a graph of percent methyl chloride conversion versus hours of time on stream, and FIG. 6 is a graph of percent selectivity to C₂-C₄ olefins versus of hours of time on stream.

TABLE 6

| | Conversion and Selectivity at | | | | |
|---|---|---|---|---|---|
| | 3 hours on stream, % | | | | |
| | | | | | C₂-C₄ |
| | CH₃Cl | C₂H₄ | C₃H₆ | C₄H₈ | olefin |
| Catalyst: | Conver- | Selec- | Selec- | Selec- | Selec- |
| Name (SAR) | sion | tivity | tivity | tivity | tivity |
| C3: HZSM-5 (278) | 57.4 | 10.9 | 39.9 | 11.5 | 62.3 |
| M: MgGaZSM-5 (278) | 93.1 | 10.4 | 45.2 | 18.8 | 74.4 |
| N: GaMgZSM-5 (278) | 99.3 | 9.5 | 22.4 | 22.4 | 84.2 |

From the data in Table 6 and FIGS. 5 and 6, it was concluded that both metal cation exchanged zeolite catalysts M and N exhibited higher methyl chloride conversion with high selectivity to C₂-C₄ olefins and deactivated significantly slower compared to the comparative catalyst C3 at 450° C., WHSV 1.35 h⁻¹ and pressure <5 psig. Furthermore, GaMgZSM-5 prepared by adding Ga to the MgZSM-5 (catalyst N) turned out to be more active, selective and stable catalyst than the MgGaZSM-5 prepared by adding Mg to GaZSM-5 (catalyst M).

1. A method for converting an alkyl halide to an olefin, the method comprising contacting a zeolite catalyst having a MFI structure with a chemical formula:
M_{y/n}H_{(x-y)}AlₓSi₍₉₆₋ₓ₎O₁₉₂
with a feed comprising an alkyl halide under reaction conditions sufficient to produce an olefin hydrocarbon product comprising C₂ to C₄ olefins,
where M is a metal cation of group IA, IIA, IIIB, IVB, VB, VIB VIIB, VIIIB, IB, IIB, IIIA or IVA, or any combination of cations thereof, and n is the valence of the charge balancing cation M, and y is 0.4≦y≦5.0, and
where the zeolite catalyst optionally contains H⁺ in addition to the metal cation.

2. The method of claim 1, wherein the zeolite catalyst has a higher alkyl halide conversion to an olefin when compared with H/ZSM-5.

3. The method of claim 1, wherein M is a Group IIA or Group IIIA metal cation or any combination thereof.

4. The method of claim 3, wherein M is magnesium (Mg), calcium (Ca), or Strontium (Sr).

5. The method of claim 4, wherein the catalyst comprises 46 to 47 wt. % silicon (Si), 0.20 to 0.30 wt. % aluminum (Al), and 0.05 to 0.25 wt. % Mg.

6. The method of claim 4, wherein the catalyst comprises 41 to 47 wt. % Si, 0.22 to 3.6 wt. % Al, and 0.08 to 0.12 wt. % Ca.

7. The method of claim 4, wherein the catalyst comprises 46 to 47 wt. % Si, 0.27 to 0.31 wt. % Al, and 0.23 to 0.27 wt. % Sr.

8. The method of claim 1, wherein M is magnesium (Mg), calcium (Ca), Strontium (Sr), cobalt (Co), copper (Cu), zinc (Zn), or gallium (Ga), or any combination thereof.

9. The method of claim 8, wherein M is a combination of Mg and Ga.

10. The method of claim 1, wherein the reaction conditions include a temperature of greater than 300° C., a weight hourly space velocity (WHSV) of greater than 0.5 h⁻¹, and a pressure of less than 5 psig, or preferably a temperature of 400 to 500° C., a weight hourly space velocity (WHSV) of 1.0 to 5.0 h⁻¹, and a pressure of less than 5 psig.

11. The method of claim 1, wherein propylene selectivity is 30% to 60% and butylene selectivity is 10 to 20%, wherein the zeolite catalyst has an alkyl halide conversion of at least 30%, at least 30 to 80%, or at least 40 to 80%, and/or wherein the combined selectivity of the C₂ to C₄ olefins is 50 to 80% at 30 to 80% alkyl halide conversion.

12. The method of claim 1, wherein the silica to alumina ratio (SAR) of the zeolite of the catalyst is 250 to 300, 270 to 290, 275 to 285, or around 278.

13. The method of claim 1, wherein the alkyl halide is methyl chloride, methyl bromide, methyl fluoride, or methyl iodide, or any combination, and wherein the feed comprises about 10 mole % or more of the methyl halide.

14. The method of claim 1, wherein the catalyst has not been subjected to a halide treatment.

15. A zeolite catalyst capable of converting a feed comprising an alkyl halide to an olefin hydrocarbon product comprising C₂ to C₄ olefins, the zeolite catalyst having a MFI structure with a chemical formula:
M_{y/n}H_{(x-y)}AlₓSi₍₉₆₋ₓ₎O₁₉₂
where M is a metal cation of group IA, IIA, IIIA. IVB, VB, VIB VIIB, IB, IIB, IIIA or IVA, or any combination of cations thereof, and n is the valence of the charge balancing cation M, and y is 0.4≦y≦5.0, and
where the zeolite catalyst optionally contains H⁺ in addition to the metal cation.

16. The zeolite catalyst of claim 15, wherein M is a Group IIA or Group IIIA metal cation or any combination thereof.

17. The zeolite catalyst of claim 16, wherein M is magnesium (Mg), calcium (Ca), or Strontium (Sr).

18. The zeolite catalyst of claim 17, wherein the catalyst comprises 41 to 47 wt. % Si, 0.20 to 3.6 wt. % Al, and 0.05 to 0.25 wt. % Mg.

19. The zeolite catalyst of claim 17, wherein the catalyst comprises 46 to 47 wt. % Si, 0.22 to 0.26 wt. % Al, and 0.08 to 0.12 wt. % Ca.

20. The zeolite catalyst of claim 17, wherein the catalyst comprises 46 to 47 wt. % Si, 0.27 to 0.31 wt. % Al, and 0.23 to 0.27 wt. % Sr.
